(19) United States
(12) Patent Application Publication
(10) Pub. No.: US 2018/0250868 A1
(43) Pub. Date: Sep. 6, 2018

(54) TUBES AND THEIR MANUFACTURE

(71) Applicant: SMITHS MEDICAL INTERNATIONAL LIMITED, Ashford (GB)

(72) Inventors: Timothy BATEMAN, Dymchurch (GB); Stephen James FIELD, Bridge (GB)

(73) Assignee: SMITHS MEDICAL INTERNATIONAL LIMITED, Ashford (GB)

(21) Appl. No.: 15/758,386

(22) PCT Filed: Aug. 26, 2016
(86) PCT No.: PCT/GB2016/000155

§ 371 Date: Mar. 8, 2018

(30) Foreign Application Priority Data
Sep. 15, 2015 (GB) 1516300.9

(51) Int. Cl. B29C 47/88; B29C 71/02; B29C 47/00; B29C 35/02; B29C 35/08; A61L 29/04; A61M 16/04

(52) U.S. Cl. CPC B29C 47/8885; B29C 71/02; B29C 47/0023; B29C 47/0035; B29C 47/0059; B29C 35/0266; B29K 2027/18; B29C 35/0805; A61L 29/041; A61M 16/0465; A61M 16/0427; B29C 2035/0838; B29C 47/881

(57) ABSTRACT

The inner cannula (3) of a tracheostomy tube includes a shaft (30) extruded of an ePTFE material. The axial strength of the shaft is increased by heating elongate portions (34 and 35) along the shaft such as by contact with a heated roller (205). The heating is sufficient to alter the structure of the material and make the heated portions (34 and 35) more rigid than the remainder of the shaft.

This invention relates to tubes of the kind having a tubular shaft of an ePTFE material.

Tracheostomy tube assemblies commonly include an outer tube and an inner tube or cannula that is a removable fit within the outer tube. The inner tube can be removed and replaced periodically to ensure that the passage through the assembly does not become blocked by secretions. This avoids the need to remove the outer tube frequently.

The inner tube presents various problems because it must be thin walled and a close fit within the outer tube so as to limit the resistance to flow of gas along the assembly. It must, however, also be sufficiently stiff to be inserted in the outer tube without buckling or kinking. A particularly suitable material for the inner cannula is PTFE or expanded PTFE (ePTFE). Cannulae made of this material can be flexed through large angles of more than 90° without kinking or significantly changing in diameter. The use of such a material in an inner cannula is described in WO94/01156 and in WO2004/101048. The Flextra tube sold by Tyco Healthcare is made of ePTFE. U.S. Pat. No. 8,419,075 describes an inner cannula of ePTFE attached with a hub at one end by an overmoulding technique. Whilst such a material has various advantages it also has a problem of poor axial stability in that it can be compressed axially by a relatively small axial force. This is a problem because, if the inner cannula cannot be freely inserted in the outer tube, such as because of a deposit or other obstruction on the inside of the outer tube, the inner cannula could be partly compressed and restrict gas flow along the assembly. It is difficult to strengthen an inner cannula of ePTFE because this material does not bond well to other materials.

It is an object of the present invention to provide an alternative tube and a method of its manufacture.

According to one aspect of the present invention there is provided a tube of the above-specified kind, characterised in that the shaft includes an elongate portion extending along a major part of its length that is rendered more rigid than the remainder of the shaft by heat treatment.

The elongate portion may extend longitudinally parallel with the axis of the shaft. The shaft may have two or more elongate portions rendered more rigid than the remainder of the shaft by heat treatment. Alternatively, the elongate portion may extend helically around the shaft. The shaft may be curved along its length.

According to another aspect of the present invention there is provided a method of making a tube including the steps of forming a shaft substantially of ePTFE, characterised in that the method includes a subsequent step of heat treating an elongate portion of the shaft extending along its length sufficiently to render the heat-treated portion more rigid than the portion that is not heat treated.

The heat treating step is preferably carried out by one or more of the following: contact by a heated roller or other member, a hot gas blade and focussed radiation such as from a laser.

According to a further aspect of the present invention there is provided a tube made by a method according to the above other aspect of the present invention.

According to a fourth aspect of the present invention there is provided a tracheostomy tube assembly including an outer tube and an inner tube according to the above one or further aspect of the present invention extending along the inside of the outer tube.

According to a fifth aspect of the present invention there is provided a machine for use in making a shaft of a tube including an extruder arranged to extrude a length of ePTFE tubing, characterised in that the machine includes heating means for applying heat to an elongate portion along the tubing extruded from the extruder sufficient to render the heated portion more rigid than the remainder of the tubing.

According to a sixth aspect of the present invention there is provided a machine for use in making a shaft of a tube including a curved mandrel on which the shaft is placed, characterised in that the machine includes two heated curved blades arranged to contact elongate portions along opposite sides of the shaft to heat the portions and render them more rigid than the remainder of the shaft.

According to a seventh aspect of the present invention there is provided a tube having a shaft made by a machine according to the above fifth or sixth aspect of the present invention.

A tracheostomy assembly with an inner cannula and its method of manufacture according to the present invention, will now be described, by way of example, with reference to the accompanying drawings, in which:

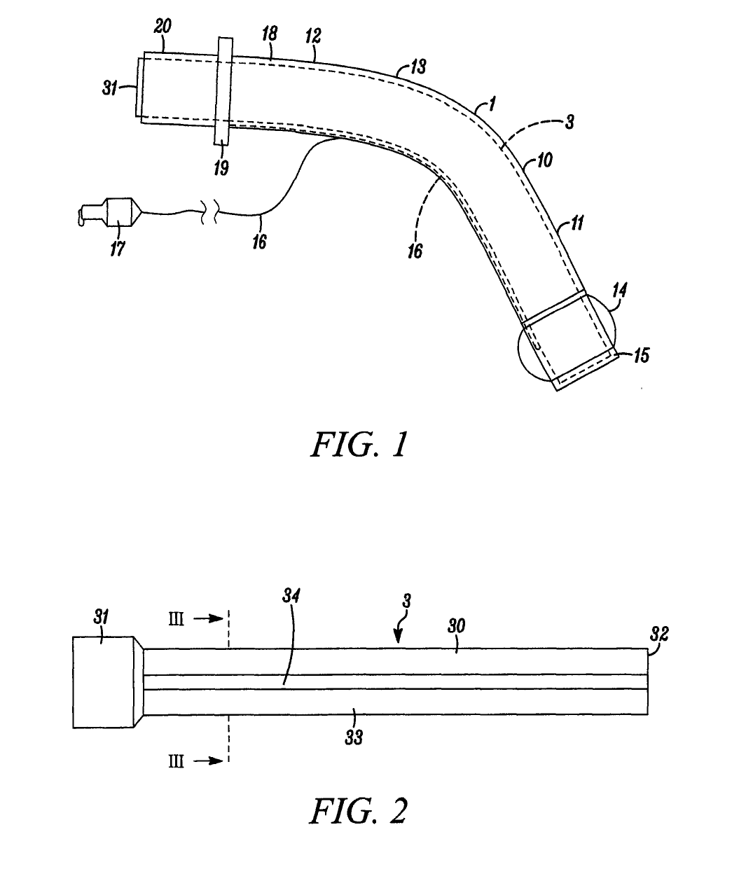

FIG. 1 shows the assembly schematically;

FIG. 2 is a plan view of the inner cannula;

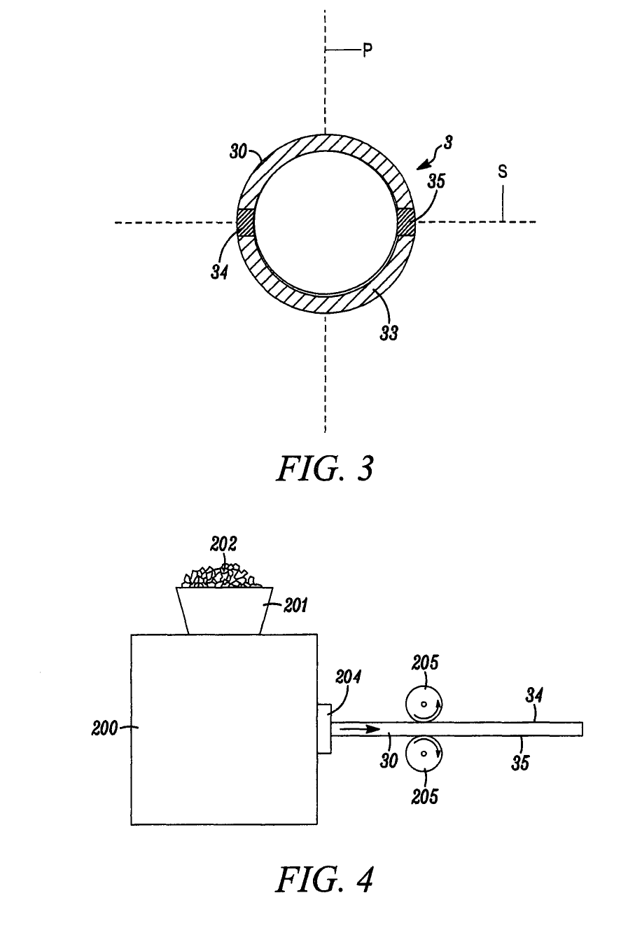

FIG. 3 is a transverse cross-sectional side elevation view of the inner cannula to an enlarged scale;

FIG. 4 illustrates a machine used in a stage in the manufacture of the inner cannula;

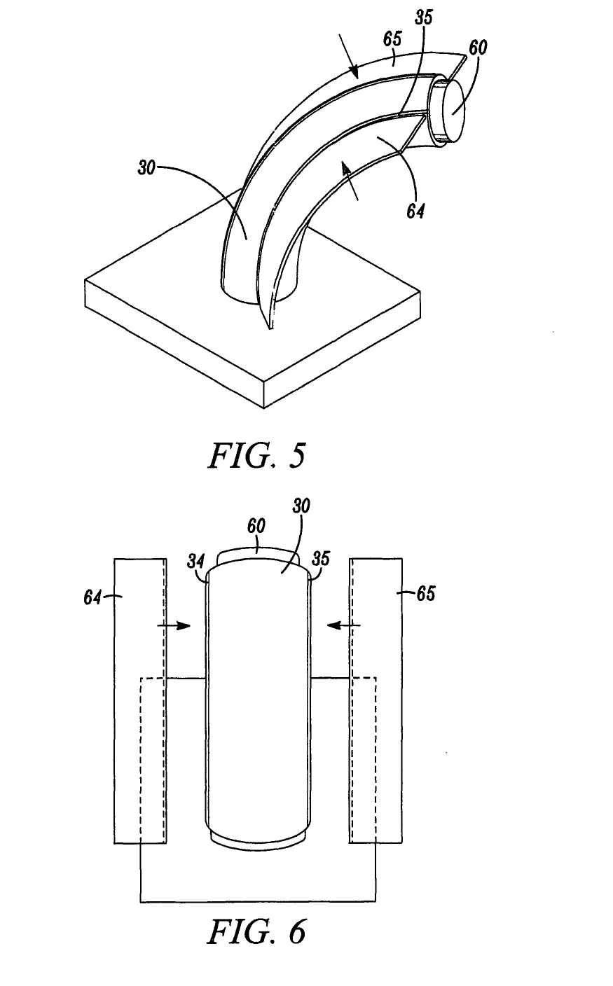

FIG. 5 is a perspective view of an alternative machine used in the manufacture of the inner cannula; and

FIG. 6 is a plan view of the machine shown in FIG. 5.

With reference first to FIG. 1, the tracheostomy tube assembly comprises an outer tube 1 and an inner tube or cannula 3, which is removable from the outer tube so that it can be periodically replaced in the usual way.

The outer tube 1 is conventional having a shaft 10 with straight forward or patient end section 11 and a rear or machine end section 12 joined by a curved section 13. Alternative outer tubes could be smoothly curved along their entire length or could be highly flexible and reinforced with a natural straight shape. A sealing cuff 14 embraces the shaft 10 close to its patient end 15. The cuff 14 can be inflated for sealing, or deflated for insertion and removal, via an inflation line 16 and a combined inflation indicator balloon and coupling 17. At its rear, machine end 18, the outer tube 1 has a flange 19 to which a tape (not shown) can be attached for securing the assembly around the neck of the patient. A hub 20 projects from the machine side of the flange 19 by which gas connection can be made to the tube 1. In use, the tube 1 extends through a surgically-made tracheostomy opening in the neck, with the patient end 15 of the tube 1 located in the trachea. The cuff 14 is inflated to form a seal between the outside of the tube and the tracheal wall so that gas flow is confined along the bore of the tube. The hub 20 at the machine end 13 of the tube 1 protrudes externally of the tracheostomy.

With reference now also to FIGS. 2 and 3, the inner tube or cannula 3 comprises a shaft 30 and a hub or machine end fitting 31. The inner cannula 3 is about 194 mm long and its shaft 30 has an internal diameter of about 8 mm with an external diameter of about 9 mm along the major part of its length. In use, the cannula 3 extends as a close sliding fit within the bore of the outer tube 1 with the patient end 32 of the cannula extending substantially level with the patient end 15 of the outer tube and with its machine end fitting 31 locating in the hub 20 of the outer tube.

The shaft 30 comprises a wall 33 made entirely of ePTFE. The ePTFE material around the major part of the wall 33 is highly flexible but along two minor portions 34 and 35 of the wall the ePTFE material is treated to make it more rigid. These minor portions 34 and 35 extend as a two straight lines or strips longitudinally parallel with the axis of the shaft 30 and diametrically opposite one another, separated by 180° as shown in FIG. 3. Alternatively, however, the shaft could just have a single more rigid strip or could have three or more strips. In further alternative arrangements the shaft could have one or more strips extending around and along the shaft in a helical fashion. Each strip 34 and 35 acts as a reinforcing or strengthening member to increases the axial stiffness of the shaft 30, reducing the risk that the shaft will be axially compressed by any axial force applied during normal use. The strips 34 and 35 still enable the shaft 30 to be bent although it gives the shaft a plane P of preferential bending that extends orthogonally to the plane S in which the two strips extend.

The strips 34 and 35 are formed by a heating process where the temperature of regions of the wall 33 of the shaft 30 is raised sufficiently to make them more rigid. A conventional tube made from ePTFE is formed by extruding an ePTFE paste and then sintering this to form a structure with PTFE fibres linked by nodes between them. The localised heating process used in the present invention acts to fuse the nodes and fibres together or, at higher temperatures, to re-melt the PTFE to form a more rigid structure in which the fibre node structure has been removed.

There are various ways in which this heat treatment process can be carried out to produce the reinforcing strips 34 and 35.

FIG. 4 shows a preliminary stage in manufacture of the inner tube or cannula 3. An extruder machine 200 has a hopper 201 of ePTFE pellets 202 and extrudes a tubular shaft 30 from its die head 204. As it emerges from the extruder 200, the shaft 30 is highly flexible around its entire circumference. The shaft 30 is given its two diametrically opposite reinforcing strips 34 and 35 by means of two thin heated rollers 205 the edges of which contact the outside of the shaft on opposite sides. The temperature of the rollers 205 and the speed that the shaft 30 is chosen such that the portion of the wall contacted by the rollers is changed in character in the manner described above. Preferably, the heating effect is arranged such that the entire thickness of the wall of the shaft 30 contacted by the edge of the rollers 205 is sufficient to effect the material changes that increase the rigidity along the two opposite strips 34 and 35.

Other alternative techniques are possible for heating the shaft to form the or each reinforcing portion or strip including other heated contact members, a hot gas blade and focussed radiation, such as from a laser.

The shaft 30 may be given a curve to enable it to fit more closely in the outer tube 1. This could be carried out by placing the shaft 30 on a mandrel 60 as shown in FIGS. 5 and 6 before any reinforcing strip has been formed along it. The mandrel 60 is curved to the desired finished shape of the inner tube or cannula 3. Two curved blades 64 and 65 are then placed against the opposite sides of the shaft so they extend along two planes parallel to and on opposite sides of the plane of curvature of the mandrel. The blades 64 and 65 are heated so that material of shaft 30 contacted by the blades is heated and thereby changed in structure to form the reinforcing strips 34 and 35. Because the reinforcing strips 34 and 35 are formed while the shaft 30 is held in a curved shape on the mandrel 60 they act to retain the shaft in the curved shape after it has been removed from the mandrel.

The inner cannula 3 is completed by attaching the hub or machine end fitting 31 to the shaft 30, which may be carried out by any conventional technique, such as by an overmoulding technique.

The curve of the completed inner cannula 3 guides the user to insert the inner cannula in the outer tube 1 with an orientation such that the reinforcing strips 34 and 35 extend along opposite sides of the curve of the cannula. This allows the remainder of the shaft 30 on the inside and outside of the curve to expand or contract as the inner cannula 3 flexes in its plane of curvature during insertion into the outer tube 1. The reinforcing strips 34 and 35 ensure that the inner tube 1 maintains the desired length so that its patient end locates at or close to the patient end 15 of the outer tube 1.

The invention is not limited to inner cannulae for tracheostomy tube assemblies but could be used with other tubes of ePTFE that need to be stiffened axially.

1-12. (canceled)

13. A tube having a tubular shaft of an ePTFE material, characterised in that the shaft includes an elongate portion extending along a major part of its length that is rendered more rigid than the remainder of the shaft by heat treatment.

14. A tube according to claim 13, characterised in that the elongate portion extends longitudinally parallel with the axis of the shaft.

15. A tube according to claim 13, characterised in that the shaft has two or more elongate portions rendered more rigid than the remainder of the shaft by heat treatment.

16. A tube according to claim 13, characterised in that the elongate portion extends helically around the shaft.

17. A tube according to claim 13, characterised in that the shaft is curved along its length.

18. A tube according to claim 13, wherein the tube is an inner tube adapted to extend along the inside of an outer tube to form a tracheostomy tube assembly.

19. A method of making a tube including the steps of forming a shaft substantially of ePTFE, characterised in that the method includes a subsequent step of heat treating an elongate portion of the shaft extending along its length sufficiently to render the heat-treated portion more rigid than the portion that is not heat treated.

20. A method according to claim 19, characterised in that the heat treating step is carried out by one or more of the following: contact by a heated roller or other member, a hot gas blade and focused radiation such as from a laser.

21. A tube made by a method including the steps of forming a shaft substantially of ePTFE, characterised in that the method includes a subsequent step of heat treating an elongate portion of the shaft extending along its length sufficiently to render the heat-treated portion more rigid than the portion that is not heat treated.
